(19) United States
(12) Patent Application Publication
(10) Pub. No.: US 2017/0065358 A1
(43) Pub. Date: Mar. 9, 2017

(54) SYSTEM PROVIDING SURGICAL IMPLANTS AND ASSOCIATED SURGICAL INSTRUMENTS

(71) Applicant: IN2BONES, Ecully (FR)

(72) Inventor: Bertrand Xavier François Gauneau, Dardilly (FR)

(73) Assignee: IN2BONES, Ecully (FR)

(21) Appl. No.: 15/254,923

(22) Filed: Sep. 1, 2016

(30) Foreign Application Priority Data
Sep. 4, 2015 (FR) FR-15 58245

(51) Int. Cl. A61B 50/30; A61B 17/80

(52) U.S. Cl. CPC A61B 50/30; A61B 17/8061; A61B 17/808; A61B 2050/3007; A61B 2050/3008

(57) ABSTRACT

A provision system of surgical implants and associated surgical instruments.
The invention concerns a provision system (1) of surgical implants intended to be attached on bone bodies of a patient, for example, his forearm, and surgical instruments intended for the implementation of these implants comprising such surgical implants and instruments in packaged form, said system (1) being characterized in that it forms a distribution unit (8) comprising:
a plurality of unitary implant holders (9, 10, 11, 12), each containing one single surgical implant, each of said unitary holders (9, 10, 11, 12) containing an implant of different configuration,
one single holder (20) of surgical instruments, containing said instruments, which are able to allow the implementation of any one of said surgical implants contained in said plurality of unitary implant holders (9, 10, 11, 12).
The provision systems of implants and osteosynthesis surgical instruments.

The present invention generally concerns the field of provision systems and methods of, on the one hand, surgical implants, in particular of osteosynthesis, in particular designed for the treatment of fractures and bone fissure fractures of bone bodies located, in particular in the forearm and, on the other hand, of surgical instruments, in particular of surgical tools intended for the implementation of such surgical implants.

The present invention concerns more particularly a provision system of surgical implants intended to be attached on bone bodies of a patient, for example, his forearm, and surgical instruments intended for the implementation of these implants, comprising such surgical implants and instruments in packaged form.

The present invention also concerns a provision method of surgical implants intended to be attached on bone bodies of a patient, for example, his forearm, and of surgical instruments intended for the implementation of these implants, from such a provision system.

During a surgical operation intended for the treatment of fractures or fissure fractures of bone bodies of a patient, it is known to provide the surgeon and his operative team with surgical implants intended to be attached on said bone bodies of the patient in order to hold in position said bone bodies relative to each other in order to, in particular allow, or at least to promote the osteosynthesis of the latter. It is also known to further provide the surgeon with the surgical instrumentation and the fastening means, in particular screws, necessary for the implementation of said implants in the body of the patient.

Thus, and by way of example, it is possible, in order to improve the reconstruction of the bones of the forearm of a patient following some types of fractures or fissure fractures in the distal end of the radius, to resort to a surgical implant, generally designated by the term [symbol] radial plate [symbol] . This type of surgical implant is most often formed by a curved plate so that the front surface of the plate is convex-shaped in order to match the shape of the end portion of the radius to be fixed. The radial plate is intended to be screwed on two parts of the radius separated by fracture so that said plate spans the fracture line and immobilizes the two parts of the radius with respect to each other in order to promote their fusion.

When a patient suffering from a fracture or a fissure fracture of the distal end of his radius is facing a surgeon in order to receive the necessary treatments and, in particular, to undergo such a distal radial plate, the latter must act quickly to carry out said treatment, without necessarily having received beforehand and exhaustively all the information necessary for the operation per se, such as the information relating to the positioning to the right or to the left of said fracture or fissure fracture and to the anatomical configuration of the radius to be treated, which may depend on the age, the sex or the corpulence of the patient.

In order to allow the surgeon to easily cope with such a situation and effectively perform the operative technique leading to the implantation of an osteosynthesis implant in the body of a patient, it has been proposed to provide and permanently keep a range of several implants of the distal radial plate type allowing covering the most common operative scenarios, namely the treatment of the fractures and the fissure fractures of the distal radius of the right forearm and of the radius of the left distal forearm, the range generally containing for each of these geometric variants at least two different anatomical configurations, respectively intended for more or less large radii.

Thus, we know provision systems, currently among the most commonly available on the market, which comprise, on the one hand, holders of implants and fastening means unitarily packaged in a sterile manner, to which are associated, one the other hand, a non-sterile and reusable instrumentation kit.

The technical and regulatory complexity and the cost of the reprocessing, in particular of cleaning, sterilization and packaging of reusable instrumentation kit is in particular high when the operation is performed within a hospital- or clinic-type healthcare center, there has been recently proposed provision systems, in which each implant of the range is packaged with an instrumentation kit in a sterile manner, into a first holder, a second holder containing a large number of fastening means of said unitarily packaged implant in a sterile manner within this second holder.

Similarly, it is also known provision systems in which each implant is packaged with an instrumentation kit and the fastening means necessary for the implementation of said implant, in a sterile manner and in a same holder.

If they actually allow overcoming the problems of sterilization and repackaging of surgical instruments and are generally satisfactory for the surgeon focusing mainly on his practitioner needs, these different known distribution systems however create huge logistical and economic problems. It is consequently apparent that they could be usefully improved.

Indeed, the different distribution systems known to date impose the storage as close as possible to the surgeon, of several holders of different implants, a surgical instrumentation kit for each of these implants, that this kit is packaged with or independently of said implants, and a set of fastening means particularly consequent.

The larger the range of implants proposed to the surgeon for a given operative surgical implantation operation is, the more the number of instruments and stored fastening means is potentially important, while the surgeon, for said given surgical operation will not immediately need all the stored instruments and fastening means.

However, the healthcare establishments and systems fall in recent years in strict budgetary policies of cost control and search for savings to cope with the general increase in healthcare expenses.

In this context, the issue of the management of the logistic and in particular the stocks of the medical devices and products is in particular crucial for all stakeholders, from the manufacturer or distributor of these devices, which generally remains owner of the devices stored in the healthcare centers until their implementation by the surgeon, to the healthcare centers which must have the means and the space necessary for the daily management of these stores and their inventory, and to the patients who directly or indirectly support the increase of the costs related to healthcare expenses.

The invention consequently aims to remedy the different drawbacks listed hereinabove and to provide a new provision system of surgical implants and associated instruments which, while allowing the surgeon to effectively cope with the most common operative scenarios, allows significantly limiting the costs relative to the surgical instrumentation.

Another object of the invention aims to provide a new provision system of surgical implants and associated instruments allowing also limiting as fairly as possible the costs relative to the fastening means of the implants in the body of the patient.

Another object of the invention is to provide a new provision system of surgical implants and associated instruments allowing a particularly safe surgical technique.

Another object of the invention ultimately aims to provide a new provision method of surgical implants and associated instruments, in particular, simple and allowing effective control by all stakeholders of the technical management and the costs relative to the storage of the distribution systems of surgical implants and associated instruments.

The objects assigned to the invention are reached using a provision system of surgical implants intended to be attached on bone bodies of a patient, for example, of his forearm, and surgical instruments intended for the implementation of these implants, comprising such surgical implants and instruments in packaged form, said system being characterized in that it forms a distribution unit comprising:
a plurality of unitary implant holders, each containing one single surgical implant, each of said unitary holders containing an implant of different configuration,
one single holder of surgical instruments, containing said instruments, which are able to allow the implementation of any one of said surgical implants contained in said plurality of unitary implant holders.

The objects assigned to the invention are also reached using a provision method of surgical implants to be attached on bone bodies of a patient, for example, of his forearm, and surgical instruments intended for the implementation of these implants, from such a provision system, characterized in that it includes the following steps:
providing a plurality of unitary implant holders, each containing one single surgical implant, each of said unitary holders containing an implant of different configuration,
providing one single holder of surgical instruments, each containing said instruments, which are able to allow the implementation of any one of said surgical implants contained in said plurality of unitary implant holders.

Other objects and advantages of the invention will further appear in more details on reading the following description as well as using the appended drawings provided by a purely explanatory and non-limiting way, in which:

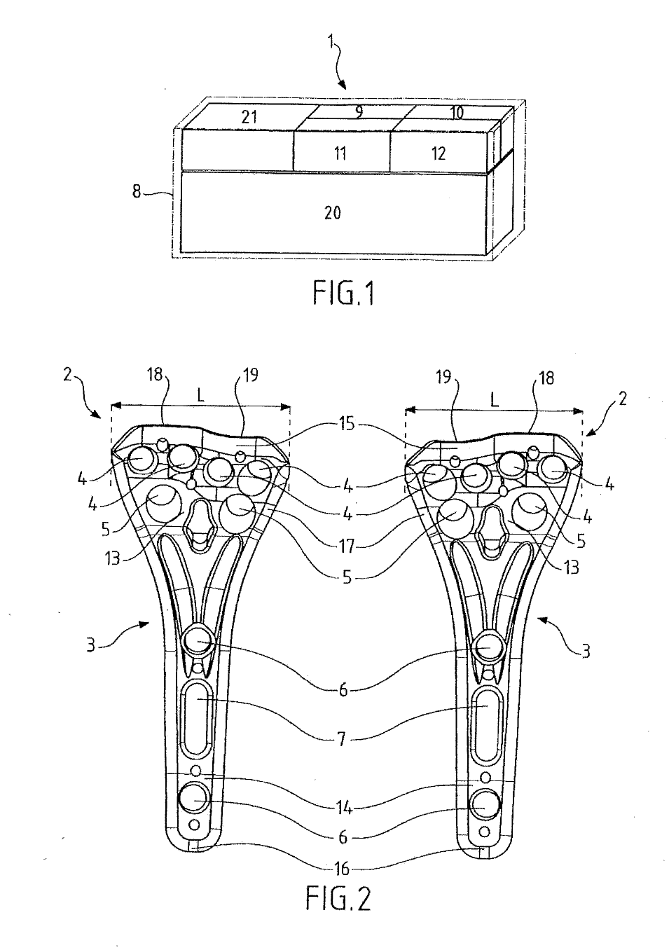

FIG. 1 schematically illustrates the provision system of the invention and the different holders that it comprises.

FIG. 2 illustrates, from the left to the right, a surgical implant of the distal radial plate type, according to configurations respectively right, that is to say for right forearm, and left, that is to say for left forearm.

The invention concerns, in its current form, a provision system 1, for example of a surgeon, with surgical implants 2 intended to be attached on bone bodies of a patient, for example, his forearm, and surgical instruments intended for the implementation of these implants 2, system 1 whose schematic non-limiting and non-exhaustive embodiment is illustrated in FIG. 1.

Said surgical implants 2, in their preferred variant shown in FIG. 2 and which is explained hereinafter, form forearm implants, in this case radial plates for fractures of the distal end of the radius of the forearm of a patient.

The invention is not however limited to a provision system 1 of forearm implants, and might concern, for example, tibial implants or palmair implants. In any case, the implants 2 are provided to be implanted in the body of the patient during a surgical operation performed, for example, under anesthesia, in particular following a lesion of a bone of the patient of the fracture or fissure fracture type, in order to treat or help to treat said lesion during an osteosynthesis process. The patient designated by the invention is preferably a human being, provided that there is no obstacle for the introduction of an implant 2 provided through the system 1 of the invention in the body of an animal to treat similar veterinary pathologies corresponding to human pathologies described herein.

Said surgical implants 2 are intended to be attached on bone bodies of a patient, that is to say provided to be introduced into the body of a patient, and to be temporarily or permanently fastened to the injured bone bodies, through fastening means in order to hold in position said bone bodies relative to each other in order, in particular, to allow, or at least to promote the osteosynthesis of the latter.

The term [symbol] fastening means [symbol] means elements, for example screws, clips or staples, which allow securing such a surgical implant 2 to said bone bodies.

In this case, such a surgical implant 2 includes a main body 3, which is intended to be attached on said bone bodies, and in which, are preferably formed, screwing openings 4, 5, 6, 7, into which are intended to be inserted fastening means, in order to secure said surgical implant 2, in particular its main body 3 with the bone bodies.

The term [symbol] bone bodies [symbol] of the bones, the cartilage, the bone or cartilage fragments, or even the tendons, or a combination thereof, preferably intended to be fused by osteosynthesis using one of said surgical implants 2, for example two fragments, or more, of the same bone, fractured separated by one or more fracture line.

According to the invention, the provision system 1 comprises such surgical implants 2 intended to be attached on bone bodies of a patient, for example, of his forearm, and surgical instruments intended for the implementation of these surgical implants 2 and instruments being in the packaged form.

Said implants 2 and surgical instruments are, indeed, preferably provided to the surgeon in packages or holders, advantageously hermetically sealed, so as to protect them from the external environment and to preserve them from any damage or contamination and unwanted dirt.

According to an important feature of the invention, said system 1 is characterized in that it forms a distribution unit 8 comprising a plurality of unitary implant holders 9, 10, 11, 12, each containing one single surgical implant 2, each of said unitary holders 9, 10, 11, 12 containing an implant 2 of different configuration.

According to a preferred embodiment of the invention, the distribution unit 8 consists of a rigid package, such as a cardboard or plastic box, structured or not, partitioned or not, as conventionally known to allow an easy and safe transport, handling, storage and distribution of the products contained in said package.

For the purpose of their provision to the surgeon by the system 1 of the invention, said distribution unit 8 comprises several implants 2, in any case more than two implants 2, which are unitarily packaged in holders, that is to say that each of said holders 9, 10, 11, 12 holds one single implant 2.

In a particularly advantageous manner, said distribution unit 8 comprises a plurality of unitary implant holders 9, 10, 11, 12, the implants 2 contained in said unitary implant holders 9, 10, 11, 12 are not strictly identical between each other but, on the contrary, preferably differing, at least partially, from each other by their respective configuration, and in particular their geometry, size and/or thickness.

Within the scope of the invention, each of said implants 2 within the unitary implant holders 9, 10, 11, 12, although of the same type and intended to the same given surgical application and, in particular, for the treatment of a given pathology even, then has a different and unique configuration of other said implants 2, that is to say for example a three-dimensional geometric conformation, an assembly mode or even a different constituent material.

Preferably, said provision system 1 preferably contains at least four unitary holders 9, 10, 11, 12 each containing one single surgical implant 2. In other words, the provision system 1 of the invention allows the provision of a surgeon, or a member of his operative team with at least four surgical implants 2, all of different configurations but suitable for the treatment of the same pathology, namely in particular the treatment of a fracture or a bone fissure fracture of a given bone, for example a forearm bone. The user is thus advantageously provided with a more or less wide choice of implants 2 allowing him to be effectively adapted to the operative situation and, in particular, to the anatomical configuration of the bone bodies of the patient to be treated. Said implants 2 being unitarily packaged in their respective holder 9, 10, 11, 12, the user can implement the implant 2 of the configuration of his choice without having to unpackage more implants 2 than necessary.

Furthermore, each of said implants 2 is advantageously packaged in a sterile manner in its holder 9, 10, 11, 12.

According to a preferred embodiment of the invention, said implants 2 are distal radial plates. In this preferred case, illustrated in FIG. 2, said implants 2 thus form forearm implants 2, intended to be attached on bone bodies, for the treatment of a fissure fracture or a fracture of the distal end of the radius of the forearm of a patient. In particular, said implants 2 are intended to be placed on the ventral part of the forearm, that is to say the part of the forearm in the extension of the palm of the hand. Of course, these implants 2 provided by the system 1 of the invention may be implemented on other bone bodies of the patient, and to treat other types of lesions without departing from the scope of the invention.

Such implants 2 of the distal radial plate type preferably include a main body 3, which is intended to be attached to the bone bodies, said main body 3 being itself preferably formed by a distal plate 13 which extends through a proximal leg 14.

Said distal plate 13 forms a portion of the main body 3 preferably located at a first end of the latter, the proximal leg 14 forming the other end in the continuity of said distal plate 13. The main body 3 preferably extends longitudinally between:
a distal edge 15, from which the distal plate 13 extends, and
a proximal edge 16, from which the proximal leg 14 extends and transversely between two lateral edges 17.

In order to be finely adapted to the bone morphology of the patient, and in particular at the end portion of the bone and the bone bodies, the distal edge 15 of the surgical implants provided by the system of the invention advantageously has a medial portion 18 and a lateral portion 19, the latter being recessed relative to said medial portion 18 so as to match the anatomical line of the bone bodies, in particular a bone of the radius of the patient. Preferably, the distal edge 15 transversely extends and is composed of two successive portions, the medial portion 18 forward relative to the lateral portion 19. The distal edge 15 has thus advantageously an [symbol] S [symbol] shape in order to limit the pain suffered by the patient. The term [symbol] medial portion [symbol] means the portion of the distal edge 15 intended to be preferably disposed on the medial side of the radius. The [symbol] lateral portion [symbol] is preferably intended to be disposed on the lateral side of the radius, opposite to the medial side.

By symmetry, and as illustrated in FIG. 2, the surgical implants 2 provided by the system 1 of the invention may be in the form of two main variants formed by a [symbol] left plate [symbol] , that is to say for a left forearm and [symbol] right plate [symbol] , that is to say for right forearm.

Each of these variants can further advantageously have sub-variants according to the maximum width L of the distal plate 13 of said implants 2, that is to say, the greatest distance which separates the lateral edges 18 of the distal plate 13, measured perpendicular to the longitudinal extension direction of the main body 3 of said implants 2. It is therefore possible to distinguish between [symbol] narrow plates [symbol] and [symbol] wide plates [symbol] , the term [symbol] narrow [symbol] is preferably applicable to radial plates whose width L of the distal plate 13 is advantageously substantially equal to 23 mm, the term [symbol] wide [symbol] , for its part, is preferably applicable to radial plates whose width L of the distal plate 13 is advantageously substantially equal to 27 mm. We have thus implants 2 in four different configuration variants: [symbol] right narrow plate [symbol] , [symbol] left narrow plate [symbol] , [symbol] right wide plate [symbol] and [symbol] left wide plate [symbol] .

The invention is not limited to a provision system 1 of forearm implants, and in particular the distal radial plates made for example hereinabove, it may perfectly concern other types of implants, and in particular osteosynthesis plates, likely to have this type of configuration variants, such as for example tibial plates.

In a particularly advantageous manner, said at least four unitary implant holders 9, 10, 11, 12 that comprises the distribution unit 8 each contain one single implant 2, preferably an osteosynthesis plate, for example a distal radial plate or a tibial plate, the latter having any of the following configurations: right narrow plate, left narrow plate, right wide plate and left wide plate.

Obviously, it would be perfectly conceivable that the implants 2 are available according to a larger variety of different configurations, for example by more finely varying the width L of the distal plate 13, by varying the length of the proximal leg 14, or even by varying the constituent materials of the main body 3 (plastic, metal, etc.) or its mechanical or physico-chemical properties (flexible/rigid, radio-transparent/radio-opaque, etc.). In this case, and always without departing from the scope of the invention, the distribution unit 8 might potentially comprise as many unitary implant holders 9, 10, 11, 12 as configurations of implants 2 considered to allow the surgeon to perform the intended surgical operation.

Consequently, according to this preferred embodiment, the surgeon thus advantageously has a range of implants 2 of various configurations allowing him to be effectively adapted to the most commonly observed scenarios of fractures or fissure fractures, mainly the fractures of the fissure fractures of the right distal radius and of the left distal radius, as well as to the anatomical variations which may have, from a patient to another, the bone bodies to be held in position relative to each other.

According to an important feature of the invention, said system 1 forms a distribution unit 8 comprising, besides said plurality of unitary implant holders 9, 10, 11, 12 as described hereinabove, only one holder 20 of surgical instruments containing such instruments, which are able to allow the implementation of any one of said surgical implants 2 contained in said plurality of unitary implant holders 9, 10, 11, 12. In other words, the surgical instruments in question are preferably packaged together in one single holder 20 of surgical instruments, and not each in a separate package, and are also advantageously designed and configured to allow the implementation of any one of said surgical implants 2 contained in said plurality of unitary implant holders 9, 10, 11, 12. The distribution unit 8 thus advantageously comprises one single holder 20 of surgical instruments holding one single kit of surgical instruments, and not a plurality of holders 20 of separate surgical instruments, each holding one or more separate surgical instruments which should be selected and regrouped prior to the completion of a given surgical operation. Thus, the system 1 is very easy to use.

Said one single holder 20 of surgical instruments can thus preferably contain one single copy of each of the instruments necessary for the implementation of an implant 2 of the unitary implant holders 9, 10, 11, 12, these instruments are moreover common to the set of implants 2 of said unitary implant holders 9, 10, 11, 12 so as to indifferently allow the implementation of one or the other of said implants 2. Thus, only the surgical instruments strictly necessary for the surgeon to the implementation of the implant 2 selected by him among the different implants 2 contained in said plurality of unitary implant holders 9, 10, 11, 12 are stored with the operative team.

Within the scope of the invention, the implementation of a surgical implant 2 using said surgical instruments cover at least the operations of placing and fastening of said surgical implant 2 in the body of a patient and, in particular, the bone bodies to be held in position relative to each other.

Said holder 20 of surgical instruments may therefore contain, for example, a screwdriver or an equivalent manual or driven screwing means, guiding pins of the positioning of the implant relative to the bone bodies, one or more piercing guides of the bone, a depth gauge and a shoe for assisting the positioning and the fastening of said implant, on said bone bodies of the patient. The latter may, however, optionally be pre-assembled on an implant 2, packaged in a unitary implant holder 9, 10, 11, 12 and therefore not be included in said holder 20 of surgical instruments.

However, without departing from the scope of the invention, such an implementation of an implant 2 may cover other steps and prior or subsequent operations to those of placing and fastening of said implant 2, such as for example the incision or the suture of the skin and the tissues, and the provision system 1 of the invention can therefore concern surgical instruments intended for other steps and operations as those of the only placing and fastening of said surgical implant 2 in the body of a patient.

Preferably, said surgical instruments provided by the distribution unit 8 are packaged in a sterile manner in their holder 20.

Thus, the surgical instrumentation necessary for the implementation of an implant 2 of said plurality of unitary implant holders 9, 10, 11, 12, is immediately operative and does not require in particular any sterilization and prior packaging operation, such operations being relatively complex and costly when performed in a hospital- or clinica-type healthcare center.

The distribution unit 8 further advantageously comprises one, and preferably one single, holder 21 of fastening means, containing fastening means, in particular fastening screws, said surgical implants 2 on said bone bodies. In other words, the fastening means in question are preferably packaged together in one single holder 21, and not each one in a separate package.

According to a preferred embodiment of the invention, said fastening means consist of locked and non-locked type fastening screws. Other fastening means may however be considered by completing or instead such screws, without departing from the scope of the invention, such as for example clipping, stapling, nailing means or similar.

Preferably, the fastening means necessary for the fastening of said surgical implants 2 on said bone bodies are identical or universal for each of the implants 2 contained in the unitary implant holders 9, 10, 11, 12 of said plurality of unitary implant holders 9, 10, 11, 12. Thus, the fastening means are advantageously able to allow the fastening of any one of said implants 2. By limiting the reference number of necessary fastening means, this versatility allows a particularly easy logistic management of the preparation and the storage of the holders 21 of fastening means.

Even more preferably, said fastening means of are packaged in an amount allowing the fastening of only one of said implants, and even more advantageously, said fastening means are packaged in a sterile manner in their holder 21. Thus the holder 21 of fastening means contains only the number of fastening means required to allow the fastening of one single implant 2 on the bone bodies, preferably increased by a few units to compensate for example the risks of loss or accidental damage by the surgeon or a member of his team during the implementation of the implant 2. Nevertheless, the number of fastening means 21 contained in the holder of fastening means, remains strictly lower than the number of fastening means which would be necessary to allow the fastening of more than one implant 2. In this manner, the surplus of unused fastening means during the surgical operation and that it should destroy, recycle or reprocess and sterilize for the purpose of future use, is in particular limited or even possibly zero, while restricting to the maximum, the number of implemented packaging elements.

In a particularly preferred manner, said implants 2, surgical instruments and fastening means are for single use only, that is to say, that they are intended to be destroyed or recycled/recovered after their use for a patient and a given surgical operation. As such, said implants 2 and surgical instruments may advantageously be made, totally or at least partially, of plastic material, for example polyether-ether-ketone (PEEK), the latter being optionally charged to confer on said implants 2 and instruments, for example, a better mechanical strength. It is however possible, without departing from the scope of the invention, that said implants 2, surgical instruments and fastening means are, on the contrary, reusable, that is to say they can, after a first use, be provided again in the distribution unit 8 after having subjected a reprocessing and proper repackaging.

Preferably, said unitary implant holders 9, 10, 11, 12, said holder 20 of surgical instruments and, advantageously also said holder 21 of fastening means are independent and separate from each other. Thus, as it will be seen hereinafter, it is advantageously possible to independently replenish the system 1 with the one and/or the other of the unitary implant holders 9, 10, 11, 12, holder 20 of surgical instruments and, if required, holder 21 of fastening means.

The invention further concerns a provision method of surgical implants 2 intended to be attached on bone bodies of a patient, for example, of his forearm, and of surgical instruments intended for the implementation of these implants 2, from a provision system 1 in accordance with the detailed description which was drawn up hereinabove.

The description hereinabove concerning the content of the unitary implant holders 9, 10, 11, 12, holders 20 of surgical instruments and holders 21 of fastening means is consequently also valid for the provision method explained hereinafter.

Such a method advantageously includes the following steps:
providing a plurality of unitary implant holders 9, 10, 11, 12, each containing one single surgical implant 2, each of said unitary holders 9, 10, 11, 12 containing an implant 2 of different configuration,
providing one single holder 20 of surgical instruments, containing said instruments, which are able to allow the implementation of any one of said surgical implants 2 contained in said plurality of unitary implant holders 9, 10, 11, 12.

Advantageously, said method further includes a step in which there is provided one, and preferably one single, holder 21 of fastening means, containing fastening means, in particular fastening screws, of said surgical implants 2 on said bone bodies.

In a particularly advantageous manner, the method of the invention comprises a replenishment step. This step, following the implementation of the implant 2 of a holder 9, 10, 11, 12 of said plurality of unitary implant holders 9, 10, 11, 12 using surgical instruments of said holder 20 of surgical instruments and fastening means of said holder 21 of fastening means, aims advantageously to replace only, by identical holders with identical content, the holders actually used by the surgeon as part of the osteosynthesis surgical operation.

Thus, in said replenishment step of the method of the invention, said provision system 1 is replenished with holders with:
a new unitary implant holder 9, 10, 11, 12, containing one single surgical implant 2, said new unitary implant holder 9, 10, 11, 12 containing an implant 2 of configuration identical to that which has been implemented,
one single new holder 20 of surgical instruments, containing said instruments, which are able to allow the implementation of any one of said surgical implants 2 contained in said plurality of unitary implant holders 9, 10, 11, 12.

Preferably, during the replenishment step, said provision system 1 is further replenished with a new holder 21 of fastening means, containing fastening means, in particular, for example fastening screws, of said surgical implants 2 on said bone bodies. Thus, at the end of said replenishment step, the surgeon has a provision system 1 complete and operational again, that is to say comprising the set of the range of different configurations of said implants 2, the surgical instruments and fastening means necessary for the implementation of said implants 2, allowing effectively performing again a new osteosynthesis surgical operation.

This replenishment step is all the more easy to perform as said implants 2, said surgical instruments and said fastening means are independently packaged.

Ultimately, the system 1 of the invention allows easily providing the surgeon with all the means and instruments necessary for performing the osteosynthesis surgical operation, the surgeon can therefore form, on demand, a complete operative kit and adapted to his needs, composed of an appropriate osteosynthesis implant 2, of surgical instruments of implementation and of the associated fastening means, while limiting the logistical and healthcare issues relating to the management of the stocks of concerned means and instruments.

1. A system (1) for the provision of surgical implants (2) intended to be attached on bone bodies of a patient, for example, his forearm, and of surgical instruments intended for the implementation of these implants (2), comprising such surgical implants (2) and instruments in packaged form, said system (1) being characterized in that it forms a distribution unit (8) comprising:
a plurality of unitary implant holders (9, 10, 11, 12), each containing one single surgical implant (2), each of said unitary holders (9, 10, 11, 12) containing an implant (2) of different configuration,
one single holder (20) of surgical instruments containing said instruments, which are able to allow the implementation of any one of said surgical implants (2) contained in said plurality of unitary implant holders (9, 10, 11, 12).

2. The system (1) according to claim 1, characterized in that it contains at least four unitary implant holders (9, 10, 11, 12) each containing one single surgical implant (2).

3. The system (1) according to claim 1, characterized in that it further comprises a holder (21) of fastening means, containing fastening means, in particular fastening screws, of said surgical implants 2 on said bone bodies.

4. The system (1) according to claim 3, characterized in that said fastening means are configured to allow the fastening of any one of said implants (2).

5. The system (1) according to claim 4, characterized in that said fastening means are packaged in an amount allowing the fastening of a single one of said implants (2).

6. The system (1) according to claim 3, characterized in that said fastening means are packaged in a sterile manner in their holder (21).

7. The system (1) according to claim 1, characterized in that each of said implants (2) is packaged in a sterile manner in its holder (9, 10, 11, 12).

8. The system (1) according to claim 1, characterized in that said at least four unitary implant holders (9, 10, 11, 12), each contains one single implant (2), the latter having any of the following configurations: right narrow plate, left narrow plate, right wide plate and left wide plate.

9. The system (1) according to claim 8, characterized in that said implants (2) are osteosynthesis plates, for example distal radial or tibial plates.

10. The system (1) according to claim 1, characterized in that said surgical instruments are packaged in a sterile manner in their holder (20).

11. The system (1) according to claim 1, characterized in that said implants 2, said surgical instruments and said fastening means are for single use only.

12. A method of provision of surgical implants (2) intended to be attached on bone bodies of a patient, for example of his forearm, and surgical instruments intended for the implementation of these implants (2), from a provision system (1) according to any one of claims 1 to 11, characterized in that it includes the following steps:
providing a plurality of unitary implant holders (9, 10, 11, 12), each containing one single surgical implant (2), each of said unitary holders (9, 10, 11, 12) containing an implant (2) of different configuration,
providing one single holder (20) of surgical instruments, containing said instruments, which are able to allow the implementation of any one of said surgical implants (2) contained in said plurality of unitary implant holders (9, 10, 11, 12).

13. The method according to claim 12, characterized in that it also includes a step in which a holder (21) of fastening means, containing fastening means, in particular fastening screws, of said surgical implants (2) on said bone bodies, is provided.

14. The method according to claim 12, characterized in that it comprises a replenishment step, following the implementation of the implant (2) of a holder (9, 10, 11, 12) of said plurality of unitary implant holders (9, 10, 11, 12) using the surgical instruments of said holder (20) of surgical instruments and fastening means of said holder (21) of fastening means, in which said replenishment step during which said provision system (1) is replenished by holders with:
a new unitary implant holder (9, 10, 11, 12), containing one single surgical implant (2), said new unitary implant holder (9, 10, 11, 12) containing one single implant (2) of identical configuration to the one which has been implemented,
a new holder (20) of surgical instruments, containing said instruments, which are able to allow the implementation of any one of said surgical implants (2) contained in said plurality of unitary implant holders (9, 10, 11, 12).

15. The method according to claim 13, characterized in that, during said replenishment step, said provision system (1) is further replenished with a new holder (21) of fastening means, containing fastening means, in particular, fastening screws, of said surgical implants (2) on said bone bodies.

16. The system (1) according to claim 2, characterized in that it further comprises a holder (21) of fastening means, containing fastening means, in particular fastening screws, of said surgical implants 2 on said bone bodies.

17. The system (1) according to claim 16, characterized in that said fastening means are configured to allow the fastening of any one of said implants (2).

18. The system (1) according to claim 16, characterized in that said fastening means are packaged in a sterile manner in their holder (21).

19. The method according to claim 13, characterized in that it comprises a replenishment step, following the implementation of the implant (2) of a holder (9, 10, 11, 12) of said plurality of unitary implant holders (9, 10, 11, 12) using the surgical instruments of said holder (20) of surgical instruments and fastening means of said holder (21) of fastening means, in which said replenishment step during which said provision system (1) is replenished by holders with:
a new unitary implant holder (9, 10, 11, 12), containing one single surgical implant (2), said new unitary implant holder (9, 10, 11, 12) containing one single implant (2) of identical configuration to the one which has been implemented,
a new holder (20) of surgical instruments, containing said instruments, which are able to allow the implementation of any one of said surgical implants (2) contained in said plurality of unitary implant holders (9, 10, 11, 12).

20. The method according to claim 14, characterized in that, during said replenishment step, said provision system (1) is further replenished with a new holder (21) of fastening means, containing fastening means, in particular, fastening screws, of said surgical implants (2) on said bone bodies.
